(19) United States
(12) Patent Application Publication
(10) Pub. No.: US 2008/0031927 A1
(43) Pub. Date: Feb. 7, 2008

(54) SOLID ORAL DOSAGE VITAMIN AND MINERAL COMPOSITIONS

(76) Inventors: Steven J. Catani, Athens, GA (US); Jacob Jae, Parsippany, NJ (US); Kenneth F. Rowe, Princeton, NJ (US); Christopher E. Szymczak, Marlton, NJ (US); Roma Vazirani, East Brunswick, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/775,899

(22) Filed: Jul. 11, 2007

Related U.S. Application Data
(60) Provisional application No. 60/807,010, filed on Jul. 11, 2006.

(51) Int. Cl. A61K 9/36; A61K 47/38; A61P 39/00; C08L 1/02

(52) U.S. Cl. 424/440; 106/170.51; 106/287.26; 106/287.35; 514/781

(57) ABSTRACT

A coating composition comprising a coating agent, a high intensity sweetener, and an acid.

### CROSS-REFERENCE TO RELATED APPLICATIONS

This application claims the benefit of priority from U.S. Provisional Patent Application No. 60/807,010, filed on Jul. 11, 2006, the content of which is incorporated by reference.

### BACKGROUND OF THE INVENTION

1. Field of the Invention

This invention relates to solid oral dosage forms, such as tablets and capsules, and methods for producing such tablets and capsules. In particular, this invention relates to coated pharmaceuticals and solid nutritional supplements in solid oral dosage forms.

2. Related Background Art

Designing pharmaceutical and nutritional supplements dosage forms that are convenient and palatable helps to facilitate patient compliance with the recommended dosing regimen. A popular solid dosage form is a tablet that is capable of being swallowed.

Tablets have been coated for a variety of reasons, including masking objectionable flavors and/or odors, protecting unstable tablet compositions, providing protection of the tablet through the stomach with enteric coatings, improving the appearance of the tablet or separating medicinal ingredients into a core segment and coating segment.

It is common practice to coat such solid dosage forms with substances, such as, film-forming polymers, fats, sugars, gelatin, and the like in order to facilitate swallowing ease, to hide an objectionable taste, and/or to provide a perceptible pleasurable taste.

Film coating pharmaceutical solid dosage forms having a solid core, such as, tablets and capsules, with thin coats of film allows efficient, controlled, uniform and reproducible coats. It is known that the use of multiple layers of coating, such as, a polymeric undercoat, a polymeric pigmented second coat, and a polymeric finish coat allows the preparation of smooth glossy tablets (U.S. Pat. No. 4,001,390).

Numerous methods for pan-coating pharmaceutical tablets have been developed and include sugar-coating techniques, solvent film coating, aqueous film coating, delayed release coating, and granule coating.

Thin film coatings, which do not alter the dissolution characteristics of the tablet, may be readily formed using aqueous film-coating processes.

Slow-dissolving coatings, pH dependent coatings, thick coatings, and sweet coatings (including sucrose or mannitol) are known. The latter are known to aid in making the tablet or capsule more palatable as well as to improve their appearance in some cases.

It is also known to include flavoring in thin-film coatings for solid oral dosage forms such as tablets and capsules.

Solid oral dosage coatings aid in tablet identification when the use of coatings containing pigments are used. Pigment addition also allows the tablets to have a more uniform and pleasing appearance. Tablet coatings comprising a colored film coating have been prepared, for example, by dispersing an anhydrous pigment suspension in a polymer solution.

Designing a dosage form with superior olfactory properties, taste, mouth feel, or other organoleptic characteristics, such as one that provides a sensory “cue” to the consumer are all known methods of obtaining a consumer-preferred product.

A need therefore remains for an economical, easy-to-swallow stable solid pharmaceutical and nutritional supplement dosage form that provides pleasant olfactory and organoleptic properties.

### SUMMARY OF THE INVENTION

The present invention is directed to a coating composition comprising (a) a coating agent, (b) a high intensity sweetener, and (c) an acid.

Yet another embodiment of this invention is directed to an orally ingestible dosage form comprising an active agent, a coating layer substantially covering the active agent, the coating layer comprising (a) a coating agent, (b) a high intensity sweetener, and (c) an acid.

The present invention includes a method of making an oral dosage form comprising the steps of (a) providing an active agent, and (b) substantially covering the active agent with a coating layer comprising a coating agent, a high intensity sweetener, and an acid.

### DETAILED DESCRIPTION OF THE INVENTION

We have unexpectedly found that the coating composition of the present invention effectively tastemasks any unpleasant taste that might otherwise have been associated with uncoated or conventional film-coated dosage forms. The user of a dosage form coated with the coating composition also experiences an enhanced flavor sensation in the throat and/or mouth. The coating composition also enhances the appeal of a particular medicine over conventional film-coated medicines so that users do not avoid taking their medicine.

A further advantage of the coating compositions of the present invention is that the resulting coated dosage form has a sweet taste without the inclusion of sugar. Not only will this improve a patient's compliance with taking the prescribed pharmaceutical, but also it will not promote tooth decay or increase caloric intake like sugar coated products. Moreover, the sugar-free coating is especially suitable for diabetic users and those restricting sugars from their diets. In addition, sugar coatings disadvantageously are relatively less stable than sucralose coatings, and thus often react with other components in the coating and discolor. Yet further, the sucralose coatings of the present invention do not provide a nutritional source for potential microbial contamination as do sugar coated products.

As used herein, the term “dosage form” applies to any solid, semi-solid, or liquid composition designed to contain a specific pre-determined amount or “dose” of a certain ingredient, for example an active ingredient as defined below. Dosage forms may include, but are not limited to: a) pharmaceutical drug delivery systems, including those for oral administration, buccal administration, rectal administration, topical or mucosal delivery, implants for subcutaneous delivery or other implanted drug delivery systems; or b) compositions for delivering minerals, vitamins and other nutraceuticals, oral care agents, flavorants, and the like. The dosage forms of the present invention are typically considered to be solid; however, they may contain liquid or semi-solid components. Suitable “solid dosage forms” of the present invention include, but are not limited to, tablets, such as, caplets, capsules, sachets, and the like. One suitable solid dosage form is an elongated tablet commonly referred to as a “caplet.” In one embodiment, the solid dosage form is an orally administered system for delivering a pharmaceutical active ingredient to the GI tract.

Dosage forms typically contain a substrate or core. As used herein, the terms “substrate” or “core” may be used interchangeably and refer to a surface or underlying support, upon which another substance at least partially resides or acts. Typically, the core is in the form of a solid such as, for example, a compressed or a molded tablet that is prepared via compression or molding. Alternatively, the core may be in the form of a semi-solid or a liquid in the finished dosage form.

“Hardness,” as used herein, describes the diametral breaking strength of either the core or the coated solid dosage form as measured by conventional pharmaceutical hardness testing equipment, such as a Schleuniger Hardness Tester. In order to compare values across different size tablets, the breaking strength must be normalized for the area of the break. This normalized value, expressed in kp/cm², is sometimes referred in the art as “tablet tensile strength.” A general discussion of tablet hardness testing is found in Leiberman et al., Pharmaceutical Dosage Forms—Tablets, Volume 2, 2nd ed., Marcel Dekker Inc., 1990, pp. 213-217 and 327-329, which is incorporated by reference herein.

“Coating composition,” as used herein, refers to a dry composition in the form of a coating on a dosage form or on a plurality of particles contained in a dosage form.

“Coating solution,” as used herein, refers to a fluid coating material in the form of a dispersion, suspension, or emulsion that is suitable for application to the surface of a substrate via, for example, spraying, dipping, or molding. Typically, the coating solution includes a solvent or liquid carrier, which is removed during processing by, for example, drying, to form the final dried coating composition.

The coating composition of the present invention includes (a) a coating agent, (b) a high intensity sweetener, and (c) an acid.

The coating agent is present in an amount from about 5 weight percent (wt. %) to about 98 wt. %, based on the total weight of the coating composition on a dry weight basis. Preferably, the coating agent is about 25 wt. % to about 95 wt. %, more preferably, about 50 wt. % to about 93 wt. %, and most preferably, about 85 wt. % to about 93 wt. %, based on the total weight of the coating composition on a dry weight basis.

The high intensity sweetener is included in the coating composition in an amount from about 0.05 wt. % to about 25 wt. %, based on the total weight of the coating composition on a dry weight basis. Preferably, the high intensity sweetener is about 0.1 wt. % to about 15 wt. %, and more preferably, about 0.5 wt. % to about 10 wt. %, based on the total weight of the coating composition on a dry weight basis.

The acid is included in the coating composition in an amount from about 0.05 wt. % to about 30 wt. %, based on the total weight of the coating composition on a dry weight basis. Preferably, the acid is about 0.1 wt. % to about 20 wt. %, and more preferably, about 0.5 wt. % to about 10 wt. %, based on the total weight of the coating composition on a dry weight basis.

In one embodiment, the coating composition of the present invention is particularly useful in the form of a coating on a solid pharmaceutical dosage form such as, for example, a swallowable tablet or caplet. In another embodiment, the coating composition of the present invention is useful in the form of a coating on a plurality of particles that contain a pharmaceutical active ingredient, and are incorporated into a pharmaceutical dosage form such as, for example, a chewable tablet.

In one embodiment, the coating composition of the present invention may be employed as a taste masking coating for active ingredient particles. Examples of taste masking coatings and methods for applying a taste masking coating onto particles are described in, for example, U.S. Pat. Nos. 4,851,226; 5,075,114, and 5,489,436, which are incorporated by reference herein. In one embodiment, the particles coated with a taste masking composition may be employed as part of a solid dosage form such as, for example, a chewable tablet. In another embodiment, the coated particles may be employed in a multiparticulate solid dosage form such as, for example, sachets, sprinkles and the like.

In embodiments in which the coating composition of the invention is incorporated as a coating on a swallowable solid dosage form, such as a swallowable tablet, the coated dosage form typically has a weight from about 50 mg to about 2000 mg, e.g., from about 100 mg to about 1600 mg, with about a 0.5 percent to about a 4 percent increase in weight relative to an uncoated dosage form.

In embodiments in which the coating composition of the invention is applied onto a particle, each individually-coated particle typically has an average diameter from about 10 microns to about 2000 microns, for example from about 50 microns to about 1000 microns or from about 100 microns to about 800 microns, whereby the thickness of the coating composition may range from about 20 microns to about 800 microns, i.e., for e.g., from about 50 microns to about 125 microns. “Water soluble” as used herein in connection with non-polymeric materials, shall mean from sparingly soluble to very soluble, i.e., not more than 100 parts water required to dissolve 1 part of the non-polymeric, water soluble solute. See Remington, “The Science and Practice of Pharmacy,” pages 208-209 (2000), which is incorporated by reference herein. “Water soluble” as used herein in connection with polymeric materials, shall mean that the polymer swells in water and can be dispersed at the molecular level to form a homogeneous dispersion.

A dosage form included in the present invention contains at least one coating layer on the substrate. Alternately, two or more layers may be employed. Colors, flavors, sweeteners, and an acid component may be combined in a single coating layer or may be in different coating layers. For example, layer 1 closest to the core may include color, layer 2 may include acid and flavor and layer 3, which surrounds layer 2 may include sweetener.

Suitable coating agents include, but are not limited to, crystallizable carbohydrates, such, as sucrose, dextrose, fructose, maltodextrin, polydextrose, and mixtures thereof and crystalizable sugar alcohols, such as, sorbitol, erythritol, lactitol, maltitol, mannitol, xylitol, and mixtures thereof and combinations of crystallizable carbohyrdrates and crystallizable sugar alcohols. Other suitable coating agents include, but are not limited to film forming polymers; waxes having a melting point of from about 5 to about 80° C. such as polyethylene glycol, bees wax, shellac wax, carnuba wax, candela wax, and microcrystalline wax; fats having a melting point less than about 80° C.; and mixtures thereof.

Any film forming polymer known in the art is suitable for use in the coating composition of the present invention. Examples of suitable film forming polymers include, but are not limited to, polyvinylalcohol (PVA), hydroxypropyl starch, hydroxyethyl starch, pullulan, methylethyl starch, carboxymethyl starch, methylcellulose, hydroxypropylcellulose (HPC), hydroxyethylmethylcellulose (HEMC), hydroxypropylmethylcellulose (HPMC), hydroxybutylmethylcellulose (HBMC), cellulose acetate (CA), cellulose acetate phthalate (CAP), carboxymethylcellulose (CMC), hydroxyethylethylcellulose (HEEC), hydroxyethylhydroxypropylmethyl cellulose (HEMPMC), starches, and polymers and derivatives and mixtures thereof.

One suitable hydroxypropylmethylcellulose compound is “HPMC 2910,” which is a cellulose ether having a degree of substitution of about 1.9 and a hydroxypropyl molar substitution of about 0.23, and containing, based upon the total weight of the compound, from about 29% to about 30% methoxyl and from about 7% to about 12% hydroxylpropyl groups. HPMC 2910 is commercially available from the Dow Chemical Company under the tradename, “Methocel E” or “Methocel E5,” which is one grade of HPMC-29 10 suitable for use in the present invention, has a viscosity of about 4 to 6 cps (4 to 6 millipascal-seconds) at 20° C. in a 2% aqueous solution as determined by a Ubbelohde viscometer. Similarly, “Methocel E6,” which is another grade of HPMC-2910 suitable for use in the present invention, has a viscosity of about 5 to about 7 cps (about 5 to about 7 millipascal-seconds) at 20° C. in a 2% aqueous solution as determined by a Ubbelohde viscometer. “Methocel E15,” which is another grade of HPMC-2910 suitable for use in the present invention, has a viscosity of about 15,000 cps (15 millipascal-seconds) at 20° C. in a 2% aqueous solution as determined by a Ubbelohde viscometer. As used herein, “degree of substitution” shall mean the average number of substituent groups attached to an anhydroglucose ring, and “hydroxypropyl molar substitution” shall mean the number of moles of hydroxypropyl per mole anhydroglucose.

As used herein, “modified starches” include starches that have been modified by crosslinking, chemically modified for improved stability, or physically modified for improved solubility properties. As used herein, “pre-gelatinized starches” or “instantized starches” refers to modified starches that have been pre-wetted, then dried to enhance their cold-water solubility. Suitable modified starches are commercially available from several suppliers such as, for example, A. E. Staley Manufacturing Company, and National Starch & Chemical Company. One suitable modified starch includes the pre-gelatinized waxy maize derivative starches that are commercially available from National Starch & Chemical Company under the tradenames, “Purity Gum” and “FilmSet,” and derivatives, copolymers, and mixtures thereof. Such waxy maize starches typically contain, based upon the total weight of the starch, from about 0 percent to about 18 percent of amylose and from about 100 percent to about 88 percent of amylopectin.

Suitable tapioca dextrins include those available from National Starch & Chemical Company under the tradename, “Crystal Gum” or “K-4484,” and derivatives thereof such as modified food starch derived from tapioca, which is available from National Starch and Chemical under the tradename, “Purity Gum 40,” and copolymers and mixtures thereof.

Examples of suitable fats include, but are not limited to hydrogenated vegetable oils such as cocoa butter, hydrogenated palm kernel oil, hydrogenated cottonseed oil, hydrogenated sunflower oil, and hydrogenated soybean oil; free fatty acids and salts thereof, and mixtures thereof.

Suitable “heat-stable, high-intensity sweeteners” shall include chemical compounds or mixtures of compounds which elicit a sweet taste at least five times sweeter than sucrose, as measured in accordance with the test method described in G.B. Patent No. 1,543,167, which is incorporated by reference herein. Typically such sweeteners are substantially free from degradants after being heated for about one hour at about 40° C. Examples of such suitable sweeteners include, but are not limited to, sucralose, neotame, saccharin, acesulfame-K, cyclamate, neohesperdine DC, stevia, thavmatin, brazzein, aspartame, and mixtures thereof.

Sucralose, which is also known as 4,1,6′-trideoxy-galactosucrose, is a heat-stable, high-intensity sweetener that may be produced in accordance with the process disclosed in U.K. Patent No. 1,544,167, and U.S. Pat. Nos. 5,136,031 and 5,498,709, which are incorporated by reference herein.

Neotame which is also known as N-(N-(3,3-dimethylbutyl)-L-a-aspartyl)-L-phenylalanine 1 methyl ester, a derivative of the dipeptide composed of the amino acids, aspartic acid and phenylalanine, is a heat-stable, high-intensity sweetener which was approved for use in the United States, July 2002 and is commercially available from The NutraSweet® Company.

Suitable acids for use in a coating layer include citric acid, malic acid, tartatic acid, ascorbic acid, fumaric acid, lactic acid, and the like. In addition, glucono delta-lactone (GDL) may be substituted for the acid.

The coating composition may also further include other ingredients, such as, based upon the total weight of the coating solution, from about 0 percent to about 30 percent of a thickener; from about 0 percent to about 15 percent plasticizers; from about 0 percent to about 1 percent preservatives such as parabens; from about 0 percent to about 5 percent opacifying agents such as titanium dioxide; and/or from about 0 percent to about 15 percent colorants. See Remington's Practice of Pharmacy, Martin & Cook, 17^{th} ed., pp. 1625-30, which is herein incorporated by reference.

Any plasticizer known in the pharmaceutical art is suitable for use in the present invention, and may include, but not be limited to polyethylene glycol; glycerin; triethyl citrate; triethyl amine; tribuyl citrate; dibutyl sebecate; vegetable oils such as castor oil; surfactants such as polysorbates, sodium lauryl sulfates, and dioctyl-sodium sulfosuccinates; propylene glycol; monoacetate of glycerol; diacetate of glycerol; triacetate of glycerol; natural gums and mixtures thereof.

Any coloring agent suitable for use in pharmaceutical application may be used in the present invention and may include, but not be limited to azo dyes, quinopthalone dyes, triphenylmethane dyes, xanthene dyes, indigoid dyes, iron oxides, iron hydroxides, titanium dioxide, natural dyes, and mixtures thereof. More specifically, suitable colorants include, but are not limited to patent blue V, acid brilliant green BS, red 2G, azorubine, ponceau 4R, amaranth, D&C red 33, D&C red 22, D&C red 26, D&C red 28, D&C yellow 10, FD&C yellow 5, FD&C yellow 6, FD&C red 3, FD&C red 40, FD&C blue 1, FD&C blue 2, FD&C green 3, brilliant black BN, carbon black, iron oxide black, iron oxide red, iron oxide yellow, titanium dioxide, riboflavin, carotenes, antyhocyanines, turmeric, cochineal extract, clorophyllin, canthaxanthin, caramel, betanin, and mixtures thereof.

Optionally, in embodiments wherein the coating agent is a film forming polymer, a fat having a melting point less than about 80° C., a wax having a melting point less than about 80° C., or a mixture thereof, the coating composition may also include, based upon the total weight of the coating composition.

An embodiment of the present invention is a tablet having three coating layers substantially surrounding the core. In this embodiment, the outer layer is a glossy layer that can be made using OPADRY® NS Clear (sodium carboxymethylcellulose, tapioca dextrin, dextrose, lecithin, sodium citrate), Opagloss2, OPADRY® II Clear (hydroxypropylmethylcellulose (HPMC), talc, medium chain triglycerides, maltodextrin, and polydextrose (all OPADRY® products are available from Colorcon)), or camauba wax with added flavor, acid, and a heat stable high intensity sweetener. This layer is typically from about 0.25 to about 3 wt% of the core or alternatively from about 0.5 to about 1 wt% of the core. The first layer substantially surrounding the core is OPADRY® II (hydroxypropylmethylcellulose (HPMC), polydextrose, titanium dioxide, talc, medium chain triglycerides, maltodextrin, and FD&C Colors) or OPADRY® NS (tapioca dextrin, titanium dioxide, hydroxypropylmethylcellulose, polyethylene glycol, dextrose monohydrate, lecithin, and FD&C colors). The middle layer or second layer from the core is OPADRY® NS or OPADRY® II with added flavors, an acid, and a heat stable high intensity sweetener.

Another embodiment of the present invention contains two coating layers substantially surrounding the core. In this embodiment, the first layer surrounding the core contains the base color that is added using a coating of OPADRY® NS or OPADRY® II. The outer layer contains OPADRY® NS or OPADRY® II with added flavor, acid, and a heat stable high intensity sweetener.

Another embodiment of the present invention contains a single coating layer that contains OPADRY® NS or OPADRY® II with added flavor, acid, and a heat stable high intensity sweetener.

It has surprisingly been found that adding acid to the coating layer increases the flavor sensation of the dosage form and does not make the coating system unstable by causing separation of the coating layers.

The vitamins and minerals are present in sufficient quantities to permit between 1 and 100% of the recommended dietary allowance of each.

The coating composition may be applied to substrates as a coating solution in the form of a liquid or liquid with a suspended solid via dipping substrates therein or spraying substrates therewith. Such coating solutions contain a solvent in an amount, based upon the total weight of the dispersion, from about 30 percent to about 99 percent, for example, from about 70 percent to about 95 percent, or from about 78 percent to about 90 percent. Examples of suitable solvents include, but are not limited to water; alcohols such as methanol, ethanol, and isopropanol; organic solvents such as methylene chloride, acetone, and the like; and mixtures thereof. In one embodiment, the solvent is water. The resulting coating solution typically possesses a solids level of, based upon the total weight of the coating solution prior to removal of the solvent, from about 1 percent to about 70 percent, e.g., from about 5 percent to about 30 percent, or from about 10 percent to about 22 percent.

In one embodiment, the coating composition of the present invention may be prepared by dissolving the sweetening agent, e.g., sucralose, in the solvent, e.g., water, through mixing. The coating agent, e.g., a film-forming polymer such as hydroxypropylmethylcellulose or a mixture of film forming polymers of hydroxypropylmethylcellulose or polyvinyl alcohol such as that commercially available from Colorcon under the tradename, “Opa-Dry NS” or “Opa-Dry II” from Colorcon); a flavor, an acid and any other remaining ingredients may then be added thereto and mixed sufficiently to form a homogeneous mixture.

Another embodiment of the present invention is directed to a solid dosage form comprised of: a) a core; b) an optional first coating layer on the surface of the core comprised of a subcoating that substantially covers the core; and c) a second coating layer substantially covering the surface of the first coating layer, with the second coating layer comprised of the coating composition of the present invention. As used herein, “substantially covers” shall mean at least about 95 percent of the surface area of the underlying substrate is covered by the given coating. For example, with respect to the first coating layer and the second coating layer, at least about 95% of the surface of the first coating layer is covered by the second coating layer.

In another embodiment, the pharmaceutical dosage form is comprised of: a) a core; b) an optional first coating layer on the surface of the core comprised of a subcoating that covers a portion of the core; and c) a second coating layer that covers a portion of the surface of the first coating layer and/or the core surface, with the second coating layer comprised of the coating composition of the present invention. As used herein, “portion” shall mean a part of the dosage form having a surface area that is equal to or less than about 95 percent of the surface area of the underlying substrate.

In one embodiment, the pharmaceutical dosage form contains a first portion, a second portion, and a plurality of outer coatings comprising the coating composition of the present invention, with the first portion having a first outer coating thereon and the second portion having a second outer coating thereon. In yet another embodiment, the second outer coating is visually distinct from the first outer coating by way of, for example, color, pattern texture, and/or the like.

The use of subcoatings is well known in the art and disclosed in, for example, United States Patent No. 3,185,626, which is incorporated by reference herein. Any composition suitable for film-coating a tablet may be used as a subcoating according to the present invention. Examples of suitable subcoatings are disclosed in U.S. Pat. Nos. 4,683,256; 4,543,370; 4,643,894; 4,828,841; 4,725,441; 4,802,924; 5,630,871; and 6,274,162, which are all incorporated by reference herein. Additional suitable subcoatings include one or more of the following ingredients: cellulose ethers such as hydroxypropylmethylcellulose, hydroxypropylcellulose, and hydroxyethylcellulose; polycarbohydrates such as xanthan gum, starch, and maltodextrin; plasticizers including for example, glycerin, polyethylene glycol, propylene glycol, dibutyl sebecate, triethyl citrate, vegetable oils such as castor oil, surfactants such as polysorbate-80, sodium lauryl sulfate and dioctyl-sodium sulfosuccinate; polycarbohydrates, pigments, and opacifiers.

In one embodiment, the subcoating may be comprised of, based upon the total weight of the subcoated tablet, from about 2 percent to about 8 percent, e.g., from about 4 percent to about 6 percent of a water-soluble cellulose ether and from about 0. 1 percent to about 1 percent castor oil, as disclosed in detail in U.S. Pat. No. 5,658,589, which is incorporated by reference herein. In another embodiment, the subcoating may be comprised of, based upon the total weight of the subcoating, from about 20 percent to about 50 percent, e.g., from about 25 percent to about 40 percent of HPMC; from about 45 percent to about 75 percent, e.g., from about 50 percent to about 70 percent of maltodextrin; and from about 1 percent to about 10 percent, e.g., from about 5 percent to about 10 percent of PEG 400.

The substrates coated with the coating composition of the present invention may contain one or more active agents. The term “active agent” is used herein in a broad sense and may encompass any material that can be carried by or entrained in the system. For example, the active agent can be a pharmaceutical, nutraceutical, vitamin, dietary supplement, nutrient, herb, dyestuff, nutritional, mineral, supplement, or the like and combinations thereof.

Any number of active agents may be contained in the dosage form. The active agents may be contained in any portion of the dosage form, e.g., in the core or substrate, in the coating composition of the invention, and/or in any additional coating. In embodiments in which active agents are contained in an additional coating, the additional coating may be, for example, a first coating layer between the substrate or core and the coating of the present invention, or may be a second coating composition residing upon a portion of the core, while the coating composition of the invention resides upon a separate portion of the core. In one embodiment, one or more active agents are contained in the core of the dosage form.

The dosage forms of the present invention contain a safe and effective amount of the active agent, which means an amount of the agent that is high enough, when administered orally, to significantly positively modify the condition to be treated or prevent an adverse or unwanted condition through short-term immediate use or repeated long-term chronic use used within the scope of sound medical judgment. The safe and effective amount of the active agent will vary with the particular condition being treated; the physical condition and age of the patient being treated; the nature of concurrent therapy, if any; the duration of the treatment; the particular carrier utilized; the specific active agent(s) employed; and the like. Typically, the active agent(s) are used in an amount, based upon the total weight of the dosage form, from about 0.001 percent to about 99.9 percent, e.g., from about 0.1 percent to about 75 percent.

The active ingredient or ingredients may be present in the dosage form in a variety of forms. For example, the active ingredient(s) may be dispersed at the molecular level, e.g., melted or dissolved, within the dosage form, or they may be in the form of particles, which in turn may be coated or uncoated. If the active ingredient is in form of particles, the particles (whether coated or uncoated) typically have an average particle size of about 1 micron to about 2000 microns. In one embodiment, such particles are crystals having an average particle size of about 1300 microns. In another embodiment, the particles are granules or pellets having an average particle size of about 50 microns to about 2000 microns, for example about 50 microns to about 1000 microns or from about 100 microns to about 800 microns.

The active agents useful herein can be selected from classes from those in the following therapeutic categories: ace-inhibitors; alkaloids; antacids; analgesics; anabolic agents; anti-anginal drugs; anti-allergy agents; anti-arrhythmia agents; antiasthmatics; antibiotics; anticholesterolemics; anticonvulsants; anticoagulants; antidepressants; antidiarrheal preparations; anti-emetics; antihistamines; antihypertensives; anti-infectives; anti-inflammatories; antilipid agents; antimanics; anti-migraine agents; antinauseants; antipsychotics; antistroke agents; antithyroid preparations; anabolic drugs; antiobesity agents; antiparasitics; antipsychotics; antipyretics; antispasmodics; antithrombotics; antitumor agents; antitussives; antiulcer agents; anti-uricemic agents; anxiolytic agents; appetite stimulants; appetite suppressants; beta-blocking agents; bronchodilators; cardiovascular agents; cerebral dilators; chelating agents; cholecystekinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypoglycemic agents; ion-exchange resins; laxatives; migraine treatments; mineral supplements; mucolytics, narcotics; neuroleptics; neuromuscular drugs; non-steroidal anti-inflammatory drugs (NSAIDs); nutritional additives; peripheral vasodilators; polypeptides; prostaglandins; psychotropics; renin inhibitors; respiratory stimulants; sedatives; steroids; stimulants; sympatholytics; thyroid preparations; tranquilizers; uterine relaxants; vaginal preparations; vasoconstrictors; vasodilators; vertigo agents; vitamins; wound healing agents; and others.

Active agents that may be used in the invention include, but are not limited to: acetaminophen; acetylsalicylic acid, including its buffered forms; acrivastine; albuterol and its sulfate; alkaline phosphatase; allantoin; aloe; aluminum acetate, carbonate, chlorohydrate and hydroxide; alprozolam; amino acids; aminobenzoic acid; amoxicillin; ampicillin; amsacrine; amsalog; anethole; ascorbic acid; astemizole; atenolol; azatidine and its maleate; bacitracin; balsam peru; BCNU (carmustine); beclomethasone diproprionate; benzophenones; benzquinamide and its hydrochloride; bethanechol; biotin; bisacodyl; bismuth subsalicylate; bomyl acetate; bromopheniramine and its maleate; buspirone; caffeine; calcium carbonate, casinate and hydroxide; camphor; captopril; cascara sagrada; cefaclor; cefadroxil; cephalexin; centrizine and its hydrochloride; cetirizine; cetylpyridinium chloride; chloramphenicol; chlorcyclizine hydrochloride; chlorhexidine gluconate; chloroxylenol; chloropentostatin; chlorpheniramine and its maleates and tannates; chlorpromazine; cholestyramine resin; choline bitartrate; chondrogenic stimulating protein; chromium, cimetidine; cinnamedrine hydrochloride; citalopram; clarithromycin; clemastine and its fumarate; clonidine; clorfibrate; codeine and its fumarate and phosphate; cortisone acetate; ciprofloxacin HCl; cyanocobalamin; cyclizine hydrochloride; cyproheptadine; danthron; dexbromopheniramine maleate; dextromethorphan and its hydrohalides; diazepam; dibucaine; dichloralphenazone; diclofen and its alkali metal sales; diclofenac sodium; digoxin; dihydroergotamine and its hydrogenates/mesylates; diltiazem; dimethicone; dioxybenzone; diphenhydramine and its citrate; diphenhydramine and its hydrochloride; divalproex and its alkali metal salts; docusate calcium, potassium, and sodium; doxycycline hydrate; doxylamine succinate; efaroxan; enalapril; enoxacin; ergotamine and its tartrate; erythromycin; estropipate; ethinyl estradiol; ephedrine; epinephrine bitartrate; erythropoietin; eucalyptol; famotidine; fenoprofen and its metal salts; ferrous fumarate, gluconate and sulfate; fexofenadine; fluoxetine; folic acid; fosphenytoin; 5-fluorouracil (5-FU); fluoxetine; flurbiprofen; furosemide; gabapentan; gentamicin; gemfibrozil; glipizide; glycerine; glyceryl stearate; granisetron; griseofulvin; growth hormone; guafenesin; hexylresorcinol; hydrochlorothiazide; hydrocodone and its tartrates; hydrocortisone and its acetate; 8-hydroxyquinoline sulfate; hydroxyzine and its pamoate and hydrochloride salts; ibuprofen; indomethacin; inositol; iron; isosorbide and its mono- and dinitrates; isoxicam; ketamine; kaolin; ketoprofen; lecithin; leuprolide acetate; lidocaine and its hydrochloride salt; lifinopril; liotrix; loperamide, loratadine; lovastatin; luteinizing hormore; LHRH (lutenizing hormone replacement hormone); magnesium carbonate, hydroxide, salicylate, and trisilicate; meclizine; mefenamic acid; meclofenamic acid; meclofenamate sodium; medroxyprogesterone acetate; methenamine mandelate; meperidine hydrochloride; metaproterenol sulfate; methscopolamine and its nitrates; methsergide and its maleate; methyl nicotinate; methyl salicylate; methsuximide; metoclopramide and its halides/hydrates; metronidazole; metoprotol tartrate; miconazole nitrate; minoxidil; morphine; naproxen and its alkali metal sodium salts; nifedipine; neomycin sulfate; niacin; niacinamide; nicotine; nicotinamide; nimesulide; nitroglycerine; nonoxynol-9; norethindrone and its acetate; nystatin; omega-3 polyunsaturated fatty acids; omeprazole; ondansetron and its hydrochloride; oxolinic acid; oxybenzone; oxtriphylline; padimate-O; paramethadione; pentastatin; pentaerythritol tetranitrate; pentobarbital sodium; perphenazine; phenelzine sulfate; phenindamine and its tartrate; pheniramine maleate; phenobarbital; phenolphthalein; phenylephrine and its tannates and hydrochlorides; phenylpropanolamine; phenytoin; pirmenol; piroxicam and its salts; polymicin B sulfate; potassium chloride and nitrate; prazepam; procainamide hydrochloride; procaterol; promethazine and its hydrochloride; propoxyphene and its hydrochloride and napsylate; pramiracetin; pramoxine and its hydrochloride salt; prochlorperazine and its maleate; propanolol and its hydrochloride; promethazine and its hydrochloride; propanolol; pseudoephedrine and its sulfates and hydrochlorides; pyridoxine; pyrolamine and its hydrochlorides and tannates; quinapril; quinidine gluconate and sulfate; quinestrol; ralitoline; ranitadine; resorcinol; riboflavin; salicylic acid; scopolamine; sesame oil; sodium bicarbonate, citrate, and fluoride; sodium monofluorophosphate; sucralfate; sulfanethoxazole; sulfasalazine; sulfur; sumatriptan and its succinate; tacrine and its hydrochloride; theophylline; terfenadine; thiethylperazine and its maleate; timolol and its maleate; thioperidone; tramadol; trimetrexate; triazolam; tretinoin; tetracycline hydrochloride; tolmetin; tolnaftate; trimethobenzamide and its hydrochloride; tripelennamine and its hydrochloride; tripolidine hydrochloride; undecylenic acid; vancomycin; verapamil HCl; vidaribine phosphate; vitamins A, B₁₋₁₂, C, D, E, and K; xylometazoline hydrochloride; zinc. Active agents may further include, but are not limited to food or herbal extracts; insoluble metal and mineral hydroxides, carbonates, oxides, polycarbophils, and salts thereof, adsorbates of active drugs on a magnesium trisilicate base and on a magnesium aluminum silicate base, and mixtures thereof.

Any of the active agents set forth above, pharmaceutically acceptable salts thereof, pharmaceutically acceptable enantiomers thereof, and mixtures thereof are also suitable for use in the present invention.

In one embodiment, the dosage form contains an active agent suitable for use in the treatment of symptoms of cough, cold, cold-like, allergy, and/or flu in a mammal. In another embodiment, the dosage form may contain guaifenesin in combination with acetaminophen and phenylephrine. In another alternative embodiment, the dosage form may be comprised of a placebo core (containing, for example, lactose and cellulose), which does not contain an active agent.

Advantageously, the inventive coating composition that substantially surrounds the core provides a tasting masking benefit. This taste masking capability is particularly useful when the active agent is comprised of vitamins, minerals, and nutritional supplements that are generally known to taste bad. In one embodiment, the dosage form contains a core that is comprised of vitamin supplements, mineral supplements, nutritional supplements, and the like. The taste masking capability of the inventive coating composition enables the core to contain up to 100% (by weight) of the desired supplement. Preferably, at least 50%, more preferably at least 60%, even more preferably at least 75%, and most preferably at least 85% by weight of the core contains the desired supplement.

The average weight gain of dried dosage form after application of the coating composition of the present invention thereto is, based upon the total weight of the dried coated dosage form, from about 0.25 percent to about 10 percent, e.g., from about 2 percent to about 4 percent. The average thickness of the dried layer of the coating composition typically is from about 30 microns to about 400 microns. However, one skilled in the art would readily appreciate without undue experimentation that the thickness of the coating composition may be varied in order to provide a smoother, easier to swallow, dosage form; to change the coating aesthetics; or to achieve a desired dissolution profile.

The present invention also includes a method of making an oral dosage form, which includes the steps of (a) providing an active agent, and (b) substantially surrounding the active agent with a coating layer comprising a coating agent, a high intensity sweetener, and an acid.

The coating composition of the present invention may be applied to the core or substrates via any methods known in the art such as, for example, spray coating, pan coating, dip coating, and molding as disclosed in, for example, McGinity, “Aqueous Polymeric Coatings for Pharmaceutical Dosage Forms” from the series “Drugs and the Pharmaceutical Sciences” (Volume 36, 1989), which is incorporated by reference herein.

In one embodiment, the coating solution may be applied to a caplet or tablet core via spraying in a pan coater using heat at a temperature sufficient to remove any solvent that may be in the coating solution. Alternatively, part or all of the core may be coated with the coating solution by dipping the substrate therein. In a further alternative embodiment, the coating solution may be sprayed onto a particle or plurality of particles, which then could be incorporated into a larger solid dosage form, e.g., compressed into a chewable tablet.

Among the noteworthy benefits from using the present invention is a noticeable improvement in the stability of the oral dosage form. This was observed during stability testing, as described in Example 5. Typically, coating compositions that include an acid component are less stable than coating compositions that do not include an acid component. However, inclusion of an acid component is sometimes desirable to improve the flavor and palatability of the oral dosage form. Oral dosage forms coated with the coating compositions of the present invention surprisingly exhibit better than expected stability results as exemplified in Example 5. Under accelerated stability conditions (40° C./75% RH) tablets coated with a coating composition containing OPADRY® NS, glucono delta-lactone, and sucralose were stable for at least 1.5 months. A second set of tablets coated with a coating composition containing OPADRY® II, citric acid, and sucralose were found to be stable for at least 3 months at 40° C./75% RH. It is generally accepted by those skilled in the art that 3 months at 40° C./75% RH is equivalent to a 24 month shelf life. The term “shelf life” is understood to refer to a product's quality attributes and/or active levels over a period of time.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

### Example 1

### Multi-Vitamin I

Prepare a premix (Premix A) of the following ingredients in a drum blender:

| | |
|---|---|
| Vitamin B12 (Cyanocobalamin 1:100 DCP) | 0.100 kg |
| Chromium (Chromium Picolinate @12.0%) | 0.016 kg |
| Calcium carbonate@37% | 2.000 kg |

Screen the following ingredients through a 16 mesh screen and add to a Gemco 10 cubic ft. V-Blender:

| | |
|---|---|
| Vitamin A (Acetate 500 m IU/g) | 0.510 kg |
| Beta Carotene 20% Beadlets @333 I.U/G | 0.625 kg |
| Ascorbic Acid @97% (Starch Free) | 9.735 kg |
| Vitamin D (Cholecalciferol 100M IU/g | 0.720 kg |
| Vitamin E (di-alpha Tocopheryl Acetate @50% | 10.020 kg |
| Vitamin K (Encap Phytonadione @1%) | 0.375 kg |
| Folate (Folic Acid Trituration @10%) | 0.750 kg |
| Vitamin B1 (Thiamine Mononitrate Powder) | 0.250 kg |
| Vitamin B2 (Ribiflavin Powder) | 0.260 kg |
| Niacin (Niacinamide Free Flow Powder) | 2.330 kg |
| Vitamin B6 (Pyridoxine Hcl Pwd @ 82.27%) | 0.370 kg |
| Biotin (Biotin Trituration in DCP@ 1%) | 0.540 kg |
| Pantothenic Acid (d-Calcium Panto @92.01%) | 1.950 kg |
| Calcium carbonate@37% | 4.595 kg |
| Iron (Ferrous Fumarate Powder @32.87%) | 8.090 kg |
| Iodine (Potassium Iodide @3%) | 0.225 kg |
| Zinc (Zinc Sulfate @36.43%) | 5.855 kg |
| Selenium (L-Selenomethionine @ 5000 mcg/g) | 1.900 kg |
| Copper (Cupric Sulfate Anhydrous @39.81%) | 0.765 kg |
| Manganese (Managanese Sulfate @32.2%) | 0.875 kg |
| Molydenum (Sodium Molybdate Trit @ 1%) | 1.140 kg |
| Potassium (Potassium Chloride @ 52.44) | 10.530 kg |
| Lutein @ 5% Bead Dry | 0.690 kg |
| Silicon Dioxide NF | 0.505 kg |
| Premix A | 2.116 kg |

Mix for 5 minutes at 13 RPM.

Stop the mixer and add the following:

| | |
|---|---|
| Calcium carbonate@37% | 68.000 kg |
| Magnesium (Magnesium Citrate Granular @16%) | 34.500 kg |

Mix for 5 minutes at 13 RPM.

Stop the mixer and add the following:

| | |
|---|---|
| Prosolv SMCC 90 | 19.359 kg |
| Klucel Nutra D Mod Cellulose | 4.005 kg |
| Croscarmellose Sodium NF | 3.505 kg |

Mix for 3 minutes at 13 RPM.

Stop the mixer. Screen the following ingredients through a 20 mesh screen and add the following:

| | |
|---|---|
| Stearic Acid NF | 3.005 kg |
| Magnesium Stearate NF | 2.005 kg |

Mix for 3 minutes at 13 RPM.

### Tablet Compression

Compress tablets on a Fette 1200 press run at about 80,000 to about 100,000 tablets per minute with a punch load of about 36.2 kN to about 48.2 kN (about 4.1 to about 5.4 tons)

### Film Coating

Add the following ingredients to a container with a propeller type mixer and mix at high speed to create a vortex.

| | | |
|---|---|---|
| OPADRY ® NS lavender 73G10225* | 4.505 | kg |
| D.I Water | 22.00 | liters |

*Blend containing Tapioca Dextrin, Titanium Dioxide, HPMC, Polyethylene Glycol, Dextrose Monohydrate, Lecithin, FD&C Blue #2, FD&C Red #40, commericially available form Colorcon as 73G10225.

Prepare a second solution using the same procedure with the following ingredients:

| | | |
|---|---|---|
| Berry Flavor | 0.365 | kg |
| Sucralose | 0.081 | kg |
| Citric Acid powder | 0.365 | kg |
| OPADRY ® NS Lavender (73G10225) | 2.005 | kg |
| D.I Water | 13.76 | liters |

Place uncoated tablets into 52 inch. Vector High Coater coating pan. Run the pan at 3.5-4.5 rpm. Spray the first solution onto the tablets with spray rate of 400-500 ml./min. Maintain the bed temperature at 42-50° C. with an inlet temperature of 75-95° C. and an exhaust temperature of 40-50° C. Keep the spray atomization pressure at 150 psi with a distance from the spray nozzle to the tablets of 10-12 in.

After the first solution has been applied, continue the application of the second solution. Once both solutions have been sprayed onto the tablets, turn off the air flow to the coater and sprinkle on the following:

| | |
|---|---|
| Carnauba Wax | 0.101 kg |

Run the coating pan for 2-5 minutes with air flow off to evenly distribute the carnauba wax.

### Example 2

### Multi-Vitamin II

Prepare a premix (Premix A) of the following ingredients in a drum blender:

| | |
|---|---|
| Vitamin B12 (Cyanocobalamin 1:100 DCP) | 0.100 kg |
| Chromium (Chromium Picolinate @12.0%) | 0.016 kg |
| Calcium carbonate@37% | 2.000 kg |

Screen the following ingredients through a 16 mesh screen and add to a Gemco 10 cubic ft. V-Blender:

| | |
|---|---|
| Vitamin A (Acetate 500 m IU/g) | 0.510 kg |
| Beta Carotene 20% Beadlets @333 I.U/G | 0.625 kg |
| Ascorbic Acid @97% (Starch Free) | 9.735 kg |
| Vitamin D (Cholecalciferol 100M IU/g | 0.720 kg |
| Vitamin E (di-alpha Tocopheryl Acetate @50% | 10.020 kg |
| Vitamin K (Encap Phytonadione @1%) | 0.375 kg |
| Folate (Folic Acid Trituration @10%) | 0.750 kg |
| Vitamin B1 (Thiamine Mononitrate Powder) | 0.250 kg |
| Vitamin B2 (Ribiflavin Powder) | 0.260 kg |
| Niacin (Niacinamide Free Flow Powder) | 2.330 kg |
| Vitamin B6 (Pyridoxine Hcl Pwd @ 82.27%) | 0.370 kg |
| Biotin (Biotin Trituration in DCP@ 1%) | 0.540 kg |
| Pantothenic Acid (d-Calcium Panto @92.01%) | 1.950 kg |
| Calcium carbonate@37% | 4.595 kg |
| Iron (Ferrous Fumarate Powder @32.87%) | 8.090 kg |
| Iodine (Potassium Iodide @3%) | 0.225 kg |
| Zinc (Zinc Sulfate @36.43%) | 5.855 kg |
| Selenium (L-Selenomethionine @ 5000 mcg/g) | 1.900 kg |
| Copper (Cupric Sulfate Anhydrous @39.81%) | 0.765 kg |
| Manganese (Managanese Sulfate @32.2%) | 0.875 kg |
| Molydenum (Sodium Molybdate Trit @ 1%) | 1.140 kg |
| Potassium (Potassium Chloride @ 52.44) | 10.530 kg |
| Lutein @ 5% Bead Dry | 0.690 kg |
| Silicon Dioxide NF | 0.505 kg |
| Premix A | 2.116 kg |

Mix for 5 minutes at 13 RPM.

Stop the mixer and add the following:

| | |
|---|---|
| Calcium carbonate@37% | 68.000 kg |
| Magnesium (Magnesium Citrate Granular@16%) | 34.500 kg |

Mix for 5 minutes at 13 RPM.

Stop the mixer and add the following:

| | |
|---|---|
| Prosolv SMCC 90 | 19.359 kg |
| Klucel Nutra D Mod Cellulose | 4.005 kg |
| Croscarmellose Sodium NF | 3.505 kg |

Mix for 3 minutes at 13 RPM.

Stop the mixer. Screen the following ingredients through a 20 mesh screen and add the following:

| | |
|---|---|
| Stearic Acid NF | 3.005 kg |
| Magnesium Stearate NF | 2.005 kg |

Mix for 3 minutes at 13 RPM.

### Tablet Compression

Compress tablets on a Fette 1200 press run at 80,000 to 100,000 tablets per minute with a punch load of 36.2 kN-48.2 kN. (4.1-5.4 tons)

### Film Coating

Add the following ingredients to a container with a propeller type mixer and mix at high speed to create a vortex.

| | | |
|---|---|---|
| OPADRY ® NS lavender 73G10225* | 5.506 | kg |
| D.I Water | 26.888 | liters |

*Blend containing Tapioca Dextrin, Titanium Dioxide, HPMC, Polyethylene Glycol, Dextrose Monohydrate, Lecithin, FD&C Blue #2, FD&C Red #40, commericially available form Colorcon as 73G10225.

Prepare a second solution using the same procedure with the following ingredients:

| | | |
|---|---|---|
| Berry Flavor | 0.183 | kg |
| Sucralose | 0.041 | kg |
| Citric Acid powder | 0.183 | kg |
| OPADRY ® NS Lavender 73G10225 | 1.003 | kg |
| D.I Water | 6.880 | liters |

Prepare a third solution using the same procedure with the following ingredients:

| | | |
|---|---|---|
| Berry Flavor | 0.183 | kg |
| Sucralose | 0.041 | kg |
| Citric Acid powder | 0.183 | kg |
| OPADRY ® NS Clear(70W19143)* | 1.014 | kg |
| D.I Water | 22.580 | liters |

*Blend containing Tapioca Dextrin, Dextrose Monohydrate, Sodium CMC, Lecithin, and Sodium Citrate which is commercially available form Colorcon as 70W19143.

Place uncoated tablets into 52 inch. Vector High Coater coating pan. Run the pan at 3.5-4.5 rpm. Spray the first solution onto the tablets with spray rate of 400-500 ml./min. Maintain the bed temperature at 42-50° C. with an inlet temperature of 75-95° C. and an exhaust temperature of 40-50° C. Keep the spray atomization pressure at 150 psi with a distance from the spray nozzle to the tablets of 10-12 in.

After the first solution has been applied, continue the application of the second solution. When application of the second solution has been completed, continue application of the third solution. After the third solution has been applied, discharge the tablets.

### Example 3

### Multi-Vitamin III

Prepare a premix (Premix A) of the following ingredients in a drum blender:

| | |
|---|---|
| Vitamin B12 (Cyanocobalamin 1:100 DCP) | 0.100 kg |
| Chromium (Chromium Picolinate @12.0%) | 0.016 kg |
| Calcium carbonate@37% | 2.000 kg |

Screen the following ingredients through a 16 mesh screen and add to a Gemco 10 cubic ft. V-Blender:

| | |
|---|---|
| Vitamin A (Acetate 500 m IU/g) | 0.510 kg |
| Beta Carotene 20% Beadlets @333 I.U/G | 0.625 kg |
| Ascorbic Acid @97% (Starch Free) | 9.735 kg |
| Vitamin D (Cholecalciferol 100M IU/g | 0.720 kg |
| Vitamin E (di-alpha Tocopheryl Acetate@50% | 10.020 kg |
| Vitamin K (Encap Phytonadione @1%) | 0.375 kg |
| Folate (Folic Acid Trituration @10%) | 0.750 kg |
| Vitamin B1 (Thiamine Mononitrate Powder) | 0.250 kg |
| Vitamin B2 (Ribiflavin Powder) | 0.260 kg |
| Niacin (Niacinamide Free Flow Powder) | 2.330 kg |
| Vitamin B6 (Pyridoxine Hcl Pwd @ 82.27%) | 0.370 kg |
| Biotin (Biotin Trituration in DCP@ 1%) | 0.540 kg |
| Pantothenic Acid (d-Calcium Panto @92.01%) | 1.950 kg |
| Calcium carbonate@37% | 4.595 kg |
| Iron (Ferrous Fumarate Powder @32.87%) | 8.090 kg |
| Iodine (Potassium Iodide @3%) | 0.225 kg |
| Zinc (Zinc Sulfate @36.43%) | 5.855 kg |
| Selenium (L-Selenomethionine @ 5000 mcg/g) | 1.900 kg |
| Copper (Cupric Sulfate Anhydrous @39.81%) | 0.765 kg |
| Manganese (Managanese Sulfate @32.2%) | 0.875 kg |
| Molydenum (Sodium Molybdate Trit @ 1%) | 1.140 kg |
| Potassium (Potassium Chloride @ 52.44) | 10.530 kg |
| Lutein @ 5% Bead Dry | 0.690 kg |
| Silicon Dioxide NF | 0.505 kg |
| Premix A | 2.116 kg |

Mix for 5 minutes at 13 RPM.

Stop the mixer and add the following:

| | |
|---|---|
| Calcium carbonate@37% | 68.000 kg |
| Magnesium (Magnesium Citrate Granular @16%) | 34.500 kg |

Mix for 5 minutes at 13 RPM.

Stop the mixer and add the following:

| | |
|---|---|
| Prosolv SMCC 90 | 19.359 kg |
| Klucel Nutra D Mod Cellulose | 4.005 kg |
| Croscarmellose Sodium NF | 3.505 kg |

Mix for 3 minutes at 13 RPM.

Stop the mixer. Screen the following ingredients through a 20 mesh screen and add the following:

| | |
|---|---|
| Stearic Acid NF | 3.005 kg |
| Magnesium Stearate NF | 2.005 kg |

Mix for 3 minutes at 13 RPM.

### Tablet Compression

Compress tablets on a Fette 1200 press run at 80,000 to 100,000 tablets per minute with a punch load of 36.2 kN-48.2 kN. (4.1-5.4 tons)

### Film Coating

Add the following ingredients to a container with a propeller type mixer and mix at high speed to create a vortex.

| | | |
|---|---|---|
| OPADRY ® II Purple 57U10235* | 5.506 | kg |
| D.I Water | 26.888 | liters |

*Blend containing, HPMC, Polydextrose, Titanium Dioxide, Talc, Maltodextrin, Medium Chain Triglycerides, FD&C Blue #2, FD&C Red #40, commercially available form Colorcon as 57U10235.

Prepare a second solution using the same procedure with the following ingredients:

| | | |
|---|---|---|
| Berry Flavor | 0.183 | kg |
| Sucralose | 0.041 | kg |
| Citric Acid powder | 0.183 | kg |
| OPADRY ® II Purple 57U10235 | 1.003 | kg |
| D.I Water | 6.880 | liters |

Prepare a third solution using the same procedure with the following ingredients:

| | | |
|---|---|---|
| Berry Flavor | 0.183 | kg |
| Sucralose | 0.041 | kg |
| Citric Acid powder | 0.183 | kg |
| OPADRY ® II Clear (57U19280)* | 1.014 | kg |
| D.I Water | 22.580 | liters |

*Blend containing Polydextrose, HPMC, Talc, Medium Chain Triglycerides, and Maltodextrin which is commercially available form Colorcon as 57U19280.

Place uncoated tablets into 52 inch. Vector High Coater coating pan. Run the pan at 3.5-4.5 rpm. Spray the first solution onto the tablets with spray rate of 400-500 ml./min. Maintain the bed temperature at 42-50° C. with an inlet temperature of 75-95° C. and an exhaust temperature of 40-50° C. Keep the spray atomization pressure at 150 psi with a distance from the spray nozzle to the tablets of 10-12 in.

After the first solution has been applied, continue the application of the second solution. When application of the second solution has been completed, continue application of the third solution. After the third solution has been applied, discharge the tablets.

### Example 4

### Easy to Swallow

A study was conducted using 149 female participants who are swallowable tablet supplement users. Each participant was given a multi-vitamin tablet coated using the coating composition of the invention and instructed to swallow the tablet. The tablet was oval shaped with dimensions of about 0.350×0.797 inches. Each participant then rated the ease of swallowing the tablet. The results are presented in the Table that follows.

| | Percent |
|---|---|
| Very easy to swallow | 73.8 |
| Somewhat easy to swallow | 16.8 |
| Neither easy nor difficult to swallow | 4.7 |
| Somewhat difficult to swallow | 2.0 |
| Very difficult to swallow | 1.3 |
| Unspecified | 1.4 |

The data shows that 90.6% of those tested found the coated Multi-Vitamin tablet “Very/Somewhat easy to swallow.” 73.8% found it “very easy to swallow” and only 2% found it “somewhat difficult to swallow.”

### Example 5

### Accelerated Stability Test for Coating System

Tablets coated with the inventive coating composition were packaged in an HDPE bottle and placed in a 40° C./75% RH (relative humidity) oven. The tablets were evaluated over a three month period of time along with a refrigerated reference control for separation of the coating layers. The following procedure was used:

Procedure:

1. Place a tablet in your mouth for 5-10 sec.
2. Evaluate for any separation of coating (mouthfeel—which is perceived as a skin/film on the tongue).
   Results:
Yes=separation of coating layers

No=no separation of coating layers

| Coating | High Intensity | | | 1 | 1.5 | 2 | 3 |
|---|---|---|---|---|---|---|---|
| System | Sweetener | Acid | Initial | month | months | months | months |
| OPADRY ® NS Color/ | Sucralose | GDL | No | No | No | Yes | Yes |
| OPADRY ® NS Clear | | | | | | | |
| OPADRY ® II Color/ | Sucralose | Citric | No | No | No | No | No |
| OPADRY ® II Clear | | | | | | | |

GDL = glucono delta-lactone

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A coating composition comprising:
(a) a coating agent;
(b) a high intensity sweetener; and
(c) an acid.

2. The coating composition of claim 1, wherein the composition further comprises a flavor.

3. The coating composition of claim 1, wherein the coating agent is hydroxypropylmethylcellulose.

4. The coating composition of claim 3, further comprising talc, maltodextrin, polydextrose, and medium chain triglycerides.

5. The coating composition of claim 1, wherein the high intensity sweetener is selected from the group consisting of sucralose, neotame, saccharin, acesulfame-K, cyclamate, neohesperdine DC, stevia, thavmatin, brazzein, aspartame, and mixtures thereof.

6. The coating composition of claim 1, wherein the acid is selected from the group consisting of citric acid, malic acid, tartatic acid, ascorbic acid, fumaric acid, lactic acid, glucono delta-lactone (GDL), and mixtures thereof.

7. The coating composition of claim 1, wherein the coating agent is present in an amount from about 5 wt. % to about 98 wt. %, based on the total wt. % of the coating composition on a dry weight basis.

8. The coating composition of claim 1, wherein the high intensity sweetener is present in an amount from about 0.05 wt. % to about 25 wt. %, based on the total wt. % of the coating composition on a dry weight basis.

9. The coating composition of claim 1, wherein the acid is present in an amount from about 0.05 wt. % to about 30 wt. %, based on the total wt. % of the coating composition on a dry weight basis.

10. An orally ingestible dosage form comprising:
an active agent;
a coating layer substantially covering the active agent;
the coating layer comprising:
(a) a coating agent,
(b) a high intensity sweetener, and
(c) an acid.

11. The dosage form of claim 10, wherein the dosage form has a shelf life of at least about 1.5 months at 40° C. and 75 % relative humidity.

12. The dosage form of claim 11, wherein the dosage form has a shelf life of at least about 3 months at 40° C. and 75 % relative humidity.

13. The dosage form of claim 10, wherein the dosage form has a shelf life of at least about 2 years.

14. The dosage form of claim 10, wherein the active agent is selected from the group consisting of vitamin supplements, mineral supplements, nutritional supplements, analgesics, anti-inflammatories, anesthetics, antihistamines, decongestants, cough suppressants, deulcents, antitussives, expectorants, and mixtures thereof.

15. The dosage form of claim 10, wherein the high intensity sweetener is sucralose.

16. A method of making an oral dosage form comprising the steps of:
(a) providing an active agent; and
(b) substantially covering the active agent with a coating layer comprising a coating agent, a high intensity sweetener, and an acid.

17. The method of claim 16, wherein the high intensity sweetener is sucralose.
